(19) United States
(12) Patent Application Publication
(10) Pub. No.: US 2012/0283798 A1
(43) Pub. Date: Nov. 8, 2012

(54) ALVEOLAR BONE REGENERATIVE APPARATUS

(76) Inventor: Toshihiko Tominaga, Tokushima (JP)

(21) Appl. No.: 13/100,244

(22) Filed: May 3, 2011

(51) Int. Cl. A61N 1/00

(52) U.S. Cl. 607/51

(57) ABSTRACT

Provided is an alveolar bone regenerative unit and an alveolar bone regenerative apparatus capable of regeneration treatment for an alveolar bone by sterilizing a periodontium and activating an osteoblast. The alveolar bone regenerative unit includes an active needle electrode of a metal electrode generating heat by power supply and a handle attached to a base of the active needle electrode. The active needle electrode is 25.0 to 41.0 mm long and 0.08 to 0.47 mm in diameter, on which a strippable coat is formed with silicon resin. Supplying power to the active needle electrode causes heat generation at a high temperature because of the thin diameter. The high temperature and electric energy can kill bacteria around a root, thereby being able to activate an osteoblast and regenerate an alveolar bone.

### BACKGROUND OF THE INVENTION

1. Field of the Invention

The present invention relates to an alveolar bone regenerative apparatus and, more particularly, to a treatment apparatus for regenerating an absorbed alveolar bone to be healthy.

2. Description of the Related Art

In a human tooth structure, a root of the tooth is surrounded and supported by an alveolar bone, and connected to a jawbone. The tooth is also surrounded by a gingiva at an upper side of the alveolar bone. When the tooth is decayed and bacteria in a nerve of the tooth reach a periphery of the root through a root canal, etiological agents are released to the alveolar bone for melting and absorbing the bone. This is called a root apex lesion.

As the alveolar bone is absorbed due to the root apex lesion, the tooth is not supported by the alveolar bone and becomes loose. According to the present treatment, tooth extraction is preformed if the alveolar bone is absorbed up to two thirds of the root or by 8 mm.

However, once the tooth extraction is performed, the tooth cannot be regenerated again. Because of this, a treatment without the tooth extraction is preferable.

Japanese Patent No. 4041165 discloses a conventional technique for treating a root apex lesion described above.

As shown in FIG. 7, this apparatus is an apparatus 10 for treating and devitalizing a vascular-nervous bundle of a decayed tooth, including a first electrode 100 in the form of needle inserted to an aperture of the tooth; a second electrode 64 attached to a patient's body; and an electric circuit electrically connected between a neutral handle 60 electrically connected to the second electrode 64 and the second electrode 64. The electric circuit includes an RF pulser generating high-frequency pulses for destroying or coagulating the vascular-nervous bundle in contact with the first electrode 100, wherein the second electrode 64 is attached to a lip of the patient, calculated is a tip position of the first electrode 100 guided by the root canal connected to a root apex of a tooth, and then high-frequency pulses are transmitted from the RF pulser to the first electrode 100.

According to the conventional technique, a pulp is devitalized only and the root apex lesion cannot be cured. That is, the bad alveolar bone cannot be regenerated.

In view of the above circumstances, an object of the present invention is to provide an alveolar bone regenerative apparatus capable of a regeneration treatment for an alveolar bone by sterilizing a periodontium and activating an osteoblast.

### SUMMARY OF THE INVENTION

An alveolar bone regenerative apparatus according to a first feature of the present invention includes an alveolar bone regenerative unit and an electro-surgical unit supplying power to the alveolar bone regenerative unit, wherein the alveolar bone regenerative unit includes an active needle electrode of a metal electrode generating heat by power supply and a handle attached to a base of the active needle electrode; the active needle electrode is 25.0 to 41.0 mm long and 0.08 to 0.47 mm in diameter, on which a coat is formed with silicon resin of which any part can be stripped off; and the electro-surgical unit supplies a current of a tone-burst wave.

An alveolar bone regenerative apparatus according to a second feature of the present invention is the first feature of the present invention, wherein the active needle electrode has a spiral shape.

An alveolar bone regenerative apparatus according to a third feature of the present invention is the first feature of the present invention, wherein the active needle electrode has a tapered shape with the base being thick and a tip being sharp.

An alveolar bone regenerative apparatus according to a fourth feature of the present invention is the first feature of the present invention, wherein the tip of the active needle electrode is 0.08 to 0.15 mm in diameter.

According to the first feature of the present invention, following effects can be obtained.

a) Since the active needle electrode is 0.08 to 0.47 mm in diameter and 25.0 to 41.0 mm long, it can pass through a root canal of a tooth and the tip thereof can reach a periphery of a root. Supplying power to the active needle electrode causes heat generation at a high temperature because of the thin diameter. The high temperature and electric energy can kill bacteria around the root and activate an osteoblast. Thus, killing the bacteria and activating the osteoblast cause a root apex lesion to enter into a healing process, thereby being able to regenerate an alveolar bone.
b) A heat treatment can be performed to a different part of the lesion by gradually changing a stripped-off part of the coat of the active needle electrode.
c) A lesion tissue can be heat-coagulated without an incision because a current supplied from the electro-surgical unit is a tone burst wave and in between waveforms is discontinuous.

According to the second feature of the present invention, the active needle electrode can be tightly coated because of the spiral shape, thereby preventing the coat from being unnoticeably stripped off.

According to the third feature of the present invention, the active needle electrode can be easily inserted into the root canal of the tooth because of the tapered shape with the tip being sharp. Moreover, damages hardly arise because of the base being thick.

According to the fourth feature of the present invention, the tip of the active needle electrode can easily pass through the root canal of the tooth because of the very sharp tip of 0.08 to 0.15 mm in diameter.

### BRIEF DESCRIPTION OF THE DRAWINGS

FIG. 1 is a diagram showing an alveolar bone regenerative unit A of an alveolar bone regenerative apparatus of the present invention;

FIG. 2 is a diagram showing a treatment used with the alveolar bone regenerative unit A of the present invention;

FIG. 3 is a diagram showing the treatment used with the alveolar bone regenerative unit A of the present invention;

FIG. 4 is a diagram showing the treatment used with the alveolar bone regenerative unit A of the present invention;

FIG. 5 is a table showing results of the treatment used with the alveolar bone regenerative apparatus of the present invention;

FIG. 6 shows photographs showing the result of the treatment used with the alveolar bone regenerative apparatus of the present invention; and

FIG. 7 is a diagram showing a conventional apparatus for treating a root apex lesion.

### DETAILED DESCRIPTION OF THE PREFERRED EMBODIMENT

An embodiment of the present invention is described with reference to the drawings.

An alveolar bone regenerative apparatus of the present invention includes an alveolar bone regenerative unit A and an electro-surgical unit supplying power to the alveolar bone regenerative unit A. The alveolar bone regenerative unit A is for regenerating an alveolar bone in which bone absorption occurs due to a lesion.

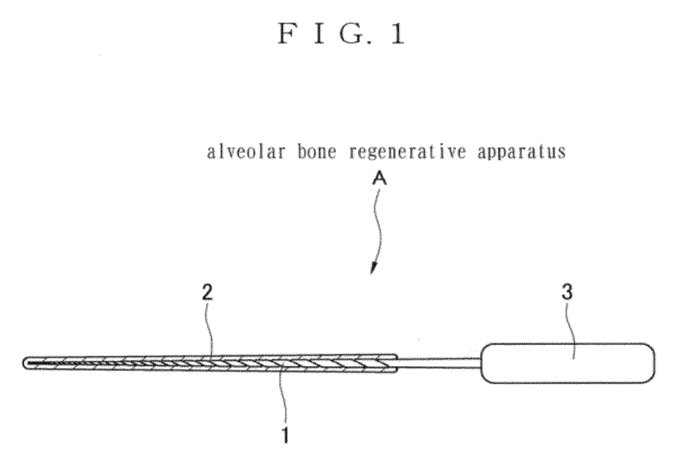

The alveolar bone regenerative unit A of one embodiment of the present invention is described with reference to FIG. 1. Reference numeral 1 indicates an active needle electrode, reference numeral 2 indicates a coat and reference numeral 3 indicates a handle.

The active needle electrode 1 is made of a metal generating heat by power supply, for which a non-corroding and high biocompatible metal is used such as stainless steel or nickel-titanium alloy.

The active needle electrode 1 is preferably 25.0 to 41.0 mm long and the most preferable is about 31.0 mm. A tip of the active needle electrode 1 can deeply reach a periphery of a root through a root canal of a tooth with this length. On the other hand, the tip thereof cannot reach the periphery of the root with a length shorter than 25.0 mm so that a regenerative treatment for the tooth cannot be performed. If the length is longer than 41.0 mm, problems arise such as a bend and a breakage of the active needle electrode 1. As a result, an operation becomes difficult.

The active needle electrode 1 is preferably 0.08 to 0.47 mm in diameter and the most preferable is about 0.10 mm. The tip of the active needle electrode 1 can easily pass through the root canal of the tooth and heat necessary for the treatment can be easily generated with this diameter. If an outside diameter is smaller than 0.08 mm, low rigidity easily causes the breakage, resulting in the difficult operation. On the other hand, if the outside diameter is larger than 0.47 mm, the tip of the active needle electrode 1 cannot easily pass through the root canal and sufficient heat cannot be obtained.

If the active needle electrode 1 has a tapered shape with a base being thick and a tip being sharp, the tip becomes flexible to easily pass through the root canal of the tooth. The taper is preferably 1/100 to 6/100 and the most preferable is about 2/100.

The active needle electrode 1 may have a needle shape with a cross section thereof being circular from the base to the tip, while it may also have a spiral shape. As for the spiral shape, a groove may be formed in a spiral shape around a needle-shaped member or a thin strip may be wound in spiral shape around the needle-shaped member.

In the case of the spiral shape, a coat 2 described below can be tightly formed, thereby being able to prevent the coat 2 from being unnoticeably stripped off.

An outside of the active needle electrode 1 is coated with the coat 2.

Preferably, a material of the coat 2 can be stripped off, for example, silicon resin. The coat 2 is formed by spreading or spraying it, or the like.

The coat 2 specifies an area to be burned in a part of the root apex lesion. That is, if the coat 2 is stripped off by about 1.0 mm to expose the active needle electrode 1, heat can be transmitted to a lesion part only by the stripped-off part. Generally, the coat 2 only at the tip of the active needle electrode 1 is stripped off by about 1 mm to generate heat from the tip. Then, a part to be treated is changed by making an inserted position of the active needle electrode 1 deep or shallow. On the other hand, a heat treatment can also be performed to a different part of the lesion by gradually changing a stripped-off part of the coat 2 and inserting the active needle electrode 1 for irradiation.

Thus, the lesion part can be treated without a large amount of current flow at once by limiting a part to be heated to a narrow area. Further, the heat cannot be generated if the tip of the active needle electrode 1 is exposed by 5 mm or more.

A handle 3 is formed at the base of the active needle electrode 1.

Used is a material that does not transmit heat to a hand at the time of power supply such as silicon, which is formed so as to be easily gripped by fingertips.

Next, usage of the electro-surgical unit supplying power to the alveolar bone regenerative unit A is described.

The electro-surgical unit is connected to a DC or AD power source (for example, a household power source of 100 V) for supplying power to the alveolar bone regenerative unit A, and includes a known electric circuit. Additionally, the electro-surgical unit is used with a counter electrode plate whose area or volume is larger than the alveolar bone regenerative unit A. Connecting the alveolar bone regenerative unit A and the counter electrode plate to the electro-surgical unit for power supply causes heat generation at the active needle electrode 1 with a small cross section, so that the heat is transmitted to the lesion part from a small stripped-off part of the coat 2.

A heat temperature of the active needle electrode 1 is preferably about 40 to 80° C. at the time of treatment. Because of this, the active needle electrode 1 made of stainless steel with the above size requires a current of 80 mA, which can be realized by the household power source of 100 V.

For a waveform of a current, a tone-burst wave is preferable to a continuous wave. A tissue is incised by a flow of a current with the continuous wave, while the tissue is heat-coagulated by a flow of a current with the discontinuous tone-burst wave. Herein, the tone-burst wave indicates a waveform of a half-wave square and each of the waveforms is discontinuous. Additionally, an arc discharge is easily generated and charge is stored near the discharge point.

If irradiation is performed for one second with 20 W±10% and 10 pulses/0.1 s (irradiation like this is called one-shot irradiation herein), for example, a temperature of the lesion part increases up to about 40 to 120° C. killing bacteria by the temperature and electric energy and activating an osteoblast. This seems to enable bone regeneration.

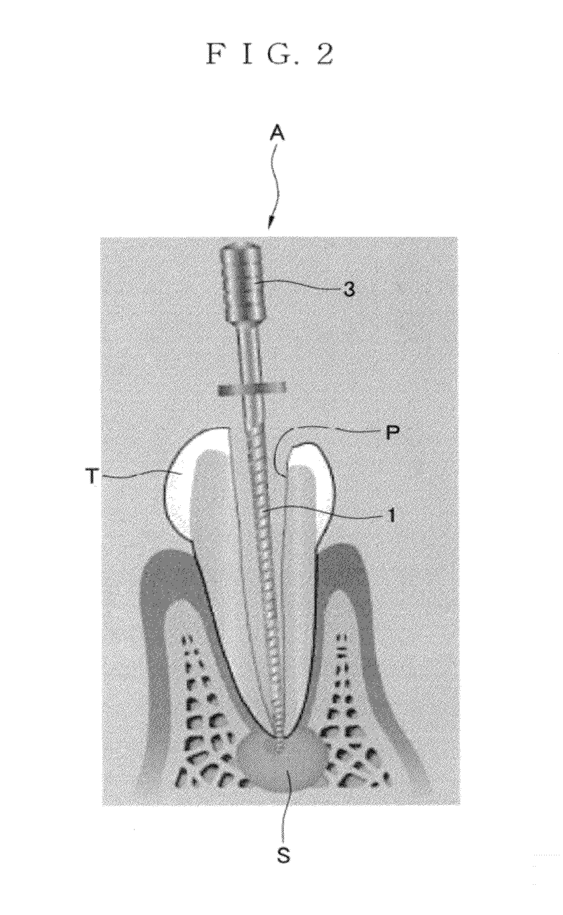

Usage of the alveolar bone regenerative unit A of the present invention is described with reference to FIGS. 2 to 4.

In FIG. 2, reference character T indicates a tooth, reference character P indicates a root canal, and reference character S indicates a lesion part. As shown in the figure, the active needle electrode 1 of the alveolar bone regenerative unit A is inserted to the lesion part S through the root canal P of the tooth T. That is, the tip of the active needle electrode 1 reaches the lesion part S. The active needle electrode 1 of the present invention can reach the lesion part S because of its long length and thin diameter, which is a big feature thereof.

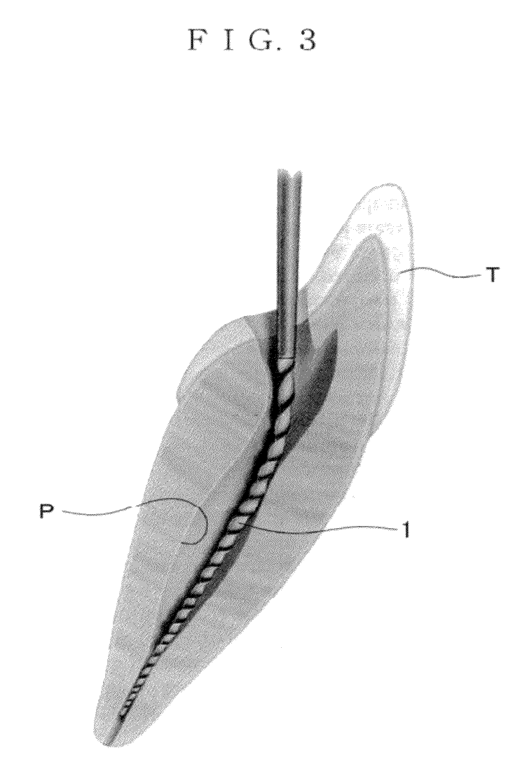

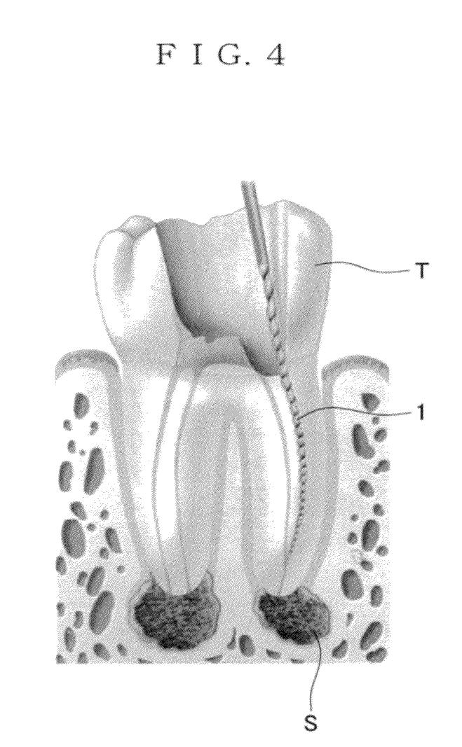

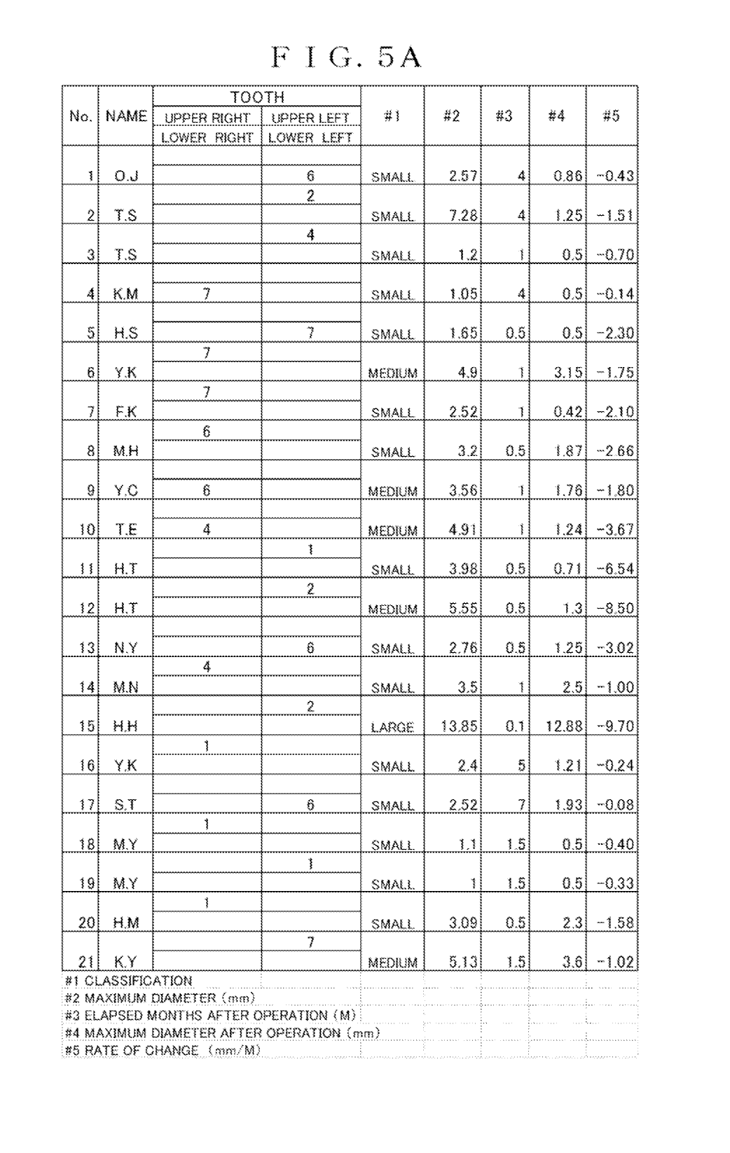

As shown in FIG. 3, if the root canal P is bent, the active needle electrode 1 can be inserted deeply to the root canal P because it is thin and easily bent. As shown in FIG. 4, if length of the root canal P to the lesion part S is long, the tip of the active needle electrode 1 can reach the lesion part S because the active needle electrode 1 has sufficient length.

Returning to FIG. 2, if a part of the coat is stripped off by aboutlmm from the tip of the active needle electrode 1, only the part transmits generated heat to the lesion part S and the lesion part S can be coagulated.

The above one-shot irradiation is performed to the lesion part (bone-absorbed part) at 2 mm intervals. In the case of a lesion part with its depth 18 mm, the one-shot irradiation is continuously performed to the part at 2.0 mm intervals and 6 to 7 shot-irradiation is performed in total. In the case of a lesion part with its depth 8 mm, the one-shot irradiation is performed four times. In the case of a lesion part with 2 mm in diameter, the one-shot irradiation is performed once.

This treatment is completed after performing the irradiation once to several times. The root canal is sealed by a filling after the irradiation. Accordingly, a patient does not have to see a doctor many times, that is, once is enough.

According to the treatment with the active needle electrode 1 of the present invention, early bone regeneration is possible because of the two reasons described below.

1) Bactericidal action

Killing bacteria in the root apex lesion suppresses bone absorption.

2) Osteoblast activation

Growth of a cell forming a bone (osteoblast) is promoted.

### Example

An effect of bone regeneration is described based on a treatment example for which the alveolar bone regenerative apparatus of the present invention is used.

### (Treatment Condition)

The alveolar bone regenerative unit A of the present invention is used for treatment to a patient who has a tooth with an intractable root apex lesion whose clinical presentation cannot be remitted by an ordinary endodontic treatment, and a tooth that cannot bear bite force because absorption of an alveolar bone is serious.

### (Results)

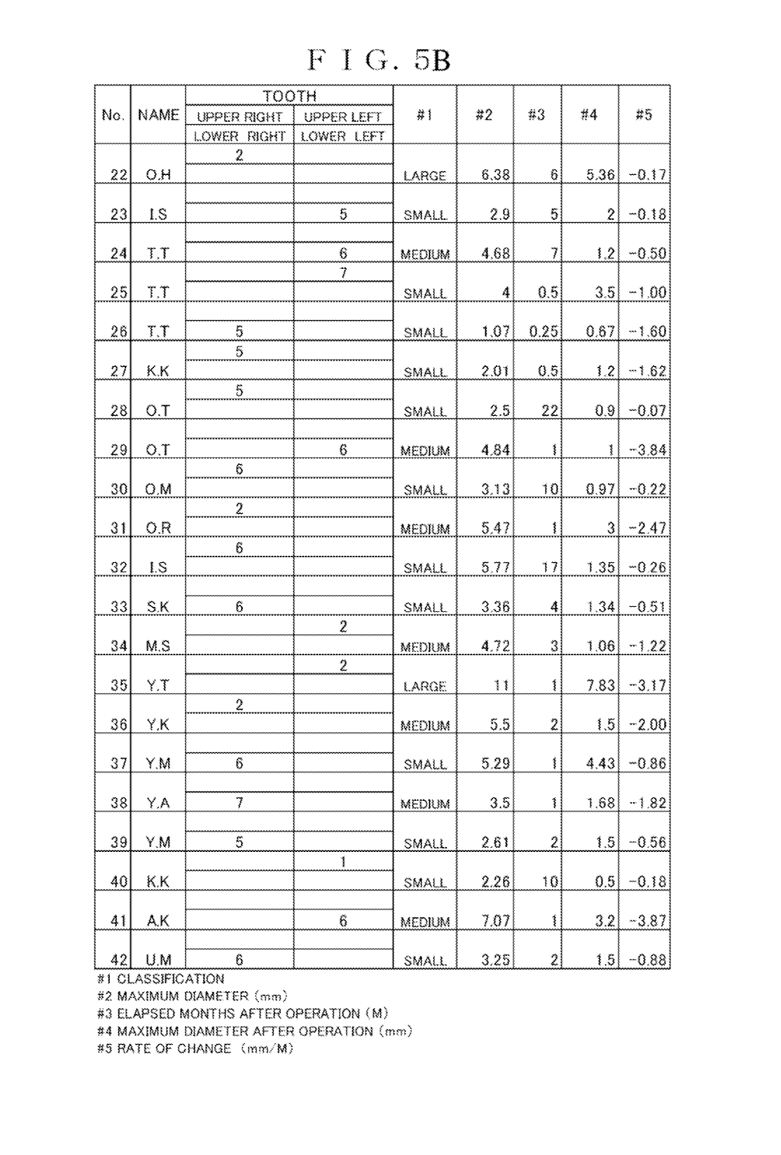

The table of FIG. 5 shows results of the treatment.

Initials of 42 participants in clinical trial are written in a name column of the table. “Lesion” indicates a cavity caused by melting a bone around a root and is classified according to a table below. Additionally, reference character M indicates the number of months elapsed in the table of FIG. 5. Each irradiation is performed with 500 kHz×1 sec and the number of shots is derived from dividing a greatest diameter of Lesion by 2.

TABLE 1
Lesion classification

None: No lesion
Small: A diameter of the lesion is twice as large as a root diameter
of 2 mm on a root apex side or less.
Medium: A diameter of the lesion is twice to three times larger than
the root diameter of 2 mm on the root apex side.
Large: A diameter of the lesion is three times larger than the root
diameter of 2 mm on the root apex side or more.

As shown in FIG. 5, all cavities (Lesions) of the 42 participants in clinical trial become smaller. Accordingly, the treatment is effective.

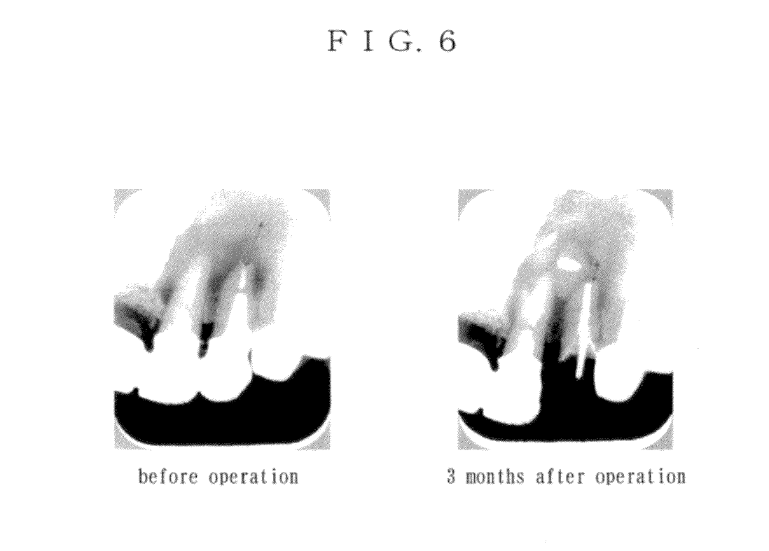

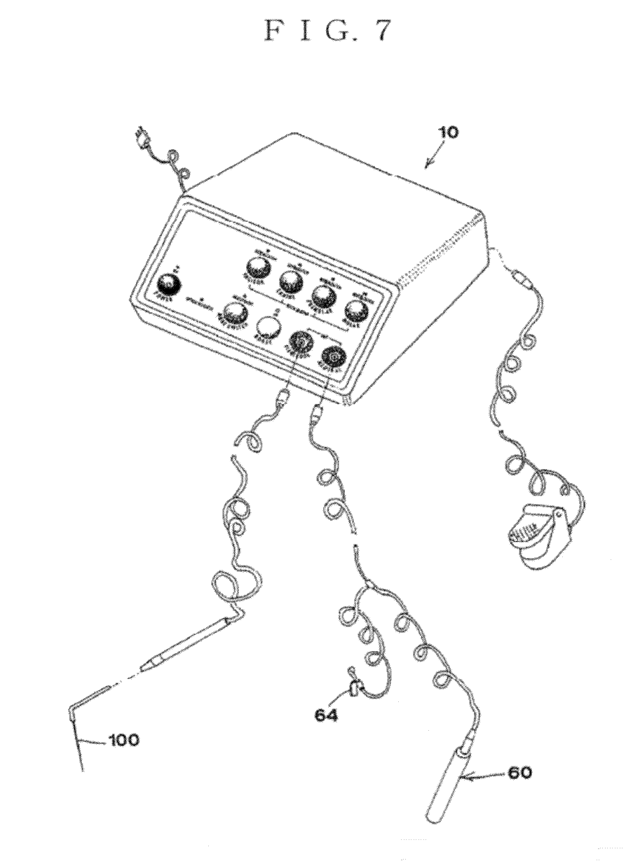

FIG. 6 shows photographs showing an alveolar bone of the No. 38 patient in FIG. 5. The photograph on the left side shows a pre-operative condition, while the photograph on the right side shows a post-operative condition after 3 months. As is evident from FIG. 6, regeneration of the alveolar bone is recognized around the root apex by 1.82 mm.

What is claimed is:

1. An alveolar bone regenerative apparatus comprising: an alveolar bone regenerative unit; and an electro-surgical unit supplying power to the alveolar bone regenerative unit,
wherein the alveolar bone regenerative unit comprises an active needle electrode of a metal electrode generating heat by power supply and a handle attached to a base of the active needle electrode; the active needle electrode is 25.0 to 41.0 mm long and 0.08 to 0.47 mm in diameter, on which a coat is formed with silicon resin of which any part can be stripped off; and the electro-surgical unit supplies a current of a tone-burst wave.

2. The alveolar bone regenerative apparatus according to claim 1, wherein the active needle electrode has a spiral shape.

3. The alveolar bone regenerative apparatus according to claim 1, wherein the active needle electrode has a tapered shape with the base being thick and a tip being sharp.

4. The alveolar bone regenerative apparatus according to claim 1, wherein the tip of the active needle electrode is 0.08 to 0.15 mm in diameter.
